(19) United States
(12) Patent Application Publication
(10) Pub. No.: US 2011/0165254 A1
(43) Pub. Date: Jul. 7, 2011

(54) Drug Delivery Using Fine Fiber Encapsulation

(75) Inventor: John A. Robertson, Chillicothe, OH (US)

(73) Assignee: KNOVATION, INC., Chillicothe, OH (US)

(21) Appl. No.: 12/983,428

(22) Filed: Jan. 3, 2011

Related U.S. Application Data
(60) Provisional application No. 61/293,047, filed on Jan. 7, 2010.

(51) Int. Cl. A61K 9/14; A61K 31/616; A61P 29/00; B82Y 5/00

(52) U.S. Cl. 424/490; 424/489; 424/501; 514/165; 427/2.14; 977/775

(57) ABSTRACT

The disclosure generally relates to a method of producing a handleable wafer of medicament powder (for example, aspirin powder) by utilizing a restraining envelope of fine water-soluble fibers, which are, upon use, quickly dissolved by bodily fluids. Such wafer would quickly provide sublingual or buccal cavity medications without significant excipients. Additional applications for such a wafer is in the prompt provision of a variety of medicaments to selected moist areas, such as, surgery or trauma sites, such as, a wound dressing.

### CROSS-REFERENCE TO RELATED APPLICATIONS

This application claims benefit of provisional application Ser. No. 61/699,984, filed on Jan. 7, 2010.

### STATEMENT REGARDING FEDERALLY SPONSORED RESEARCH

Not applicable.

### BACKGROUND

### Motivation

This inventor previously received U.S. Pat. No. 6,516,950, entitled, “Credit Card-Size Carrier for a Medicament”, wherein a credit card sized carrier was disclosed, which contains a dose of emergency medication (e.g., aspirin). That patent did not teach a specific method whereby the handleable wafer-like dose of medicament would be produced. The present invention generally relates to an inventive method of producing such a wafer from a powder by utilizing a restraining envelope of fine water-soluble fibers, which are, upon use, quickly dissolved by bodily fluids. Such a wafer would quickly deliver sublingual or buccal medications without additional excipient binders or coatings. Additional applications for such a wafer include, for example, the prompt provision of a variety of medicaments to selected moist areas, such as, for example, surgery or trauma sites.

### Introduction

The disclosure will be illustrated specifically with respect to aspirin as an example drug medication in powder form and not by way of limitation, as the novel delivery system described here is further applicable to use with many other drugs in powder form.

It would be desirable to have a dose of aspirin within credit card sized holder for a medicament as described in U.S. Pat. No. 6,516,950, which would assist in the speedy sublingual (under the tongue) or buccal (cheek) intake. This would be especially important if the aspirin were taken during a medical emergency where a standard dry pill or powder might be swallowed or inhaled incorrectly.

There is some prior art related to sublingual aspirin. For example, Perovitch, U.S. Pat. No. 5,683,721, “Galenic Preparation of Therapeutic Composition Ccomprising Aspirin”, suggests dissolving the active ingredient (aspirin) in ethanol and in effect wetting a food support (sorbitol, sugar, or mannitol) and drying to infuse the food support with microparticles of the aspirin. This process is repeated several times due to the low aspirin deposition per cycle. An adjuvant, PEG (polyethylene glycol), is provided as a lubricant for the pill molding process. The PEG also is noted for adding a hydrophilic coating for dissolving in saliva. A Perovitch disclosed representative formulation follows:

| Amount (mg) | Ingredient |
|---|---|
| 100 | Acetylsalicylic acid (Rhone-Poulenc) - |
| | dissolved in ethanol 4x amount by weight |
| 125 | PEG 6000 (Polyethylene Glycol) |
| 10 | Pulverant anhydrous citric acid (pH |
| | corrector) |
| 950 | Sorbitol w60 support (Roquette) |
| 15 | Xanthane gelling agent (Kelco/Sunofi - |
| | Bio Industrie) |
| 7 | Aspertarn sweetener (Searle-PCI) |
| 10 | Orange aroma (Roberer) |
| 20 | Na Stearyl fumarate lubricant |

The net result is a low percentage of aspirin (approximately 27 mg in a 300 mg tablet or wafer). This low percentage of active ingredient would make it impractical to use a similar formulation for the credit card sized holder for a medicament, as described in U.S. Pat. No. 6,516,950^{1.} Additionally, the formulation proposed by Perovitch is not quickly dissolved sublingually (under the tongue). ¹ The cavity described in U.S. Pat. No. 6,516,950 is approx 1 mm×65 mm×40 mm=2600 mm³ holding 2600 mg at density=1 gm/cm³. At 10% active ingredient, this would be 260 mg of aspirin—less than the typical (albeit tablet to stomach) dose for cardiac emergencies.

Attempts have been made to electrospin fine fibers from water-based fluids (e.g., PVA) containing medicaments^{2.} The resultant fluids typically contain less than about 10% of the medicament and the dried fiber mat then typically contains less than 5% medicament. ² Drug-loaded ultrafine poly(vinyl alcohol) fibre mats prepared by electrospinning, Chunxue Zhang, Xiaoyan Yuan, Lili Wu, Jing Sheng, e-Polymers 2005 no. 072.

### BRIEF SUMMARY

Rather than utilize a formulation which has a relatively large percentage of absorbent fillers into which a low solubility active ingredient (e.g., aspirin dissolved in ethanol) can be infused or by electrospinning fibers containing small percentages of medicament particles, this disclosure utilizes a high percentage of active ingredient (typically in a powdered form) in a layer enclosed, on at least one side, by a porous, fibrous web of (water) soluble fine fibers. The high surface area and low mass of the fine soluble fibers ensures that they will be rapidly dissolved upon use.

The following description teaches the fine fiber coating of an individual medicament wafer. It will be appreciated by one skilled in the art, that the production process could be accomplished on a continuous web followed by the cutting of individual wafers. Also, while the disclosure will be with specific reference to PVA fibers, it will be appreciated that these are illustrative only and not limitative of the disclosure. Any water-soluble (actually, body fluid soluble) material that is approved by appropriate regulatory authorities for human (animal in the case of veterinarian use) ingestion and can formed in fibers having diameters of between 50 nanometers and 20 micrometers is suitable for use herein.

Similarly, while powdered aspirin is specifically used herein, it will be appreciated that this is illustrative only and not limitative of the disclosure. In practice, any medicament or drug that is approved for human ingestion in powdered form can be used in accordance with the instant disclosure.

### BRIEF DESCRIPTION OF THE DRAWINGS

For a fuller understanding of the nature and advantages of the present device and process, reference should be had to the following detailed description taken in connection with the accompanying drawings, in which:

FIG. 1a depicts a plan view of a single wafer of medicament powder generally at 1 shown as individual powder particles 2 (not to scale). Such powder may be continuous or discontinuous.

FIG. 1b depicts a cross-sectional view through line 1b of the single wafer in FIG. 1a (not to scale). Thickness 3 may, for example, be 0.5 mm.

FIG. 2a depicts a plan view of the single wafer after a web of fibers 7 is applied to the topside of the wafer. This is not to scale. In the preferred construction, fine fibers 7 are much smaller than the medicament particles and the openings between fine fibers 8 are smaller than the medicament particles to effectively “trap” the powder. Again, the layer of medicament need not be uniform in density or continuity; inasmuch areas of less dense particles and/or areas with no particles are possible.

FIG. 2b is a cross-sectional view along line 2b of the single wafer in FIG. 2a.

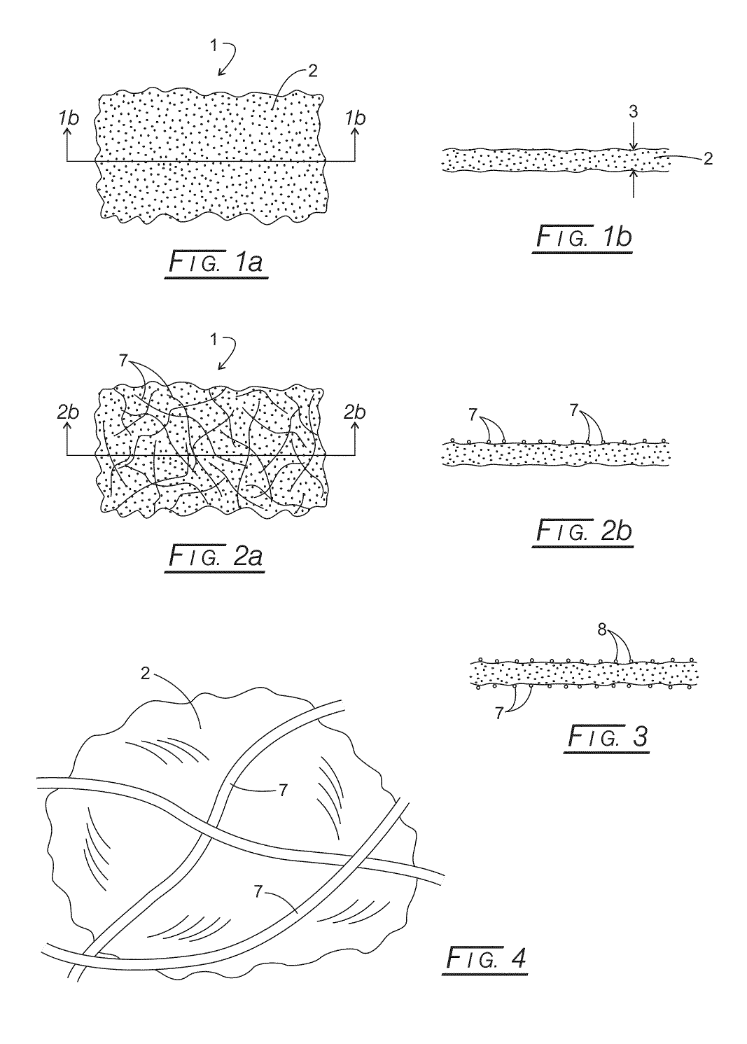

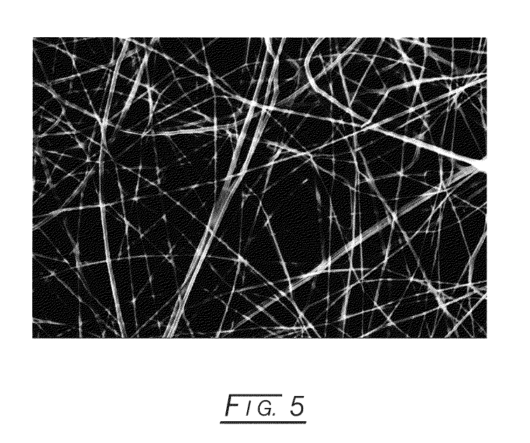

The one-sided web coated wafer is may be flipped (for example, on a carrier ribbon—details not shown) so that a second web, as shown as 8 in FIG. 3, traps the medicament particles between the two webs, thereby creating a handleable wafer. It may be advantageous to have areas of little to no medicament particles to encourage bonding of the webs from both sides of the medicament layer. This would create a firmer and more handleable produce wafer. Use of multiple layers on one or both sides, optionally, of different thickness, density, and/or composition additional may find advantage in accordance with precepts of this disclosure.

FIG. 4 depicts the web fibers and medicament particles close to microscopic scale. Note that medicament particles 2 are much larger than the openings between the fine fibers 7.

FIG. 5 is a photomicrograph of a PVA electrospun web having fibers less than 1 micron in diameter and representative openings in the web being less than 3-micron square area.

These drawings will be described in further detail below.

### DETAILED DESCRIPTION

Considering fine fibers of diameter DF (micrometers) and specific weight of SP (grams per cubic centimeter), noting that for a deposition of C (grams), fibers are produced with a total fiber of length:

$$TL ( cm ) = \frac{( C * 4.0 {e}^{+ 8} )}{π * DF * DF * SP}$$

For example, a 1 mg fibers (C=0.001 gm) of 1 gm/cc (SP=1) density having 1 micron diameter would be 12.7 e⁺⁴ centimeters long and could web a 1 cm square wafer 127,000 times or 63,500 times per side. To a reasonable (orthogonal) approximation, the “pores” between fibers would on average be 1/31,750 cm=3.1 microns square. Such small pores would be capable of retaining even fine ground medicament powders.

The above example illustrates how the retaining web excipient becomes a very small percentage of the total wafer. Suppose that the 1 cm×1 cm wafer powder is 0.5 mm thick, giving a powder volume of 0.05 cc, which at 1 gm/cc, is 50 mg of active medicament. The high surface area fine fiber web is then 1 mg/50 mg=2% of the wafer and will be quickly dissolved by saliva or bodily fluids.

By comparison, a continuous wafer coating film only 10 microns thick would require 20 mg of excipient, which then becomes 40% (by weight) of the wafer. The disclosed fine fiber web entrainment of the medicament clearly enables the body (e.g., saliva or wound fluids) to more easily and more rapidly acquire the active medication.

The disclosed fine fiber web can be formed by a variety of spraying or mechanical shearing processes. The preferred fiber web application method utilizes electrospinning, because electrospinning is capable of producing strong, continuous fibers below 1 micron in diameter. A photomicrograph of a typical electrospun fiber web (PVA) is shown in FIG. 5.

The fiber web, which constrains the medication, should be water-soluble and FDA approved for inclusion in pharmaceuticals. Electrospun polyvinyl alcohol, for example, fibers are a representative web. FIG. 5 shows a photomicrograph of a PVA electrospun web having fibers less than 1 micron in diameter and representative openings in the web being less than 3-micron square area.

While the composition and process have been described with reference to various embodiments, those skilled in the art will understand that various changes may be made and equivalents may be substituted for elements thereof without departing from the scope and essence of the disclosure. In addition, many modifications may be made to adapt a particular situation or material to the teachings of the disclosure without departing from the essential scope thereof. Therefore, it is intended that the disclosure not be limited to the particular embodiments disclosed, but that the disclosure will include all embodiments falling within the scope of the appended claims. In this application all units are in the metric system and all amounts and percentages are by weight, unless otherwise expressly indicated. Also, all citations referred herein are expressly incorporated herein by reference.

I claim:

1. A method of providing a handleable wafer of medicament, which comprises:
coating a layer of medicament powder on at least one side with fine water-soluble fibers, said fibers having diameters of between 50 nanometers and 20 micrometers.

2. The method of claim 1, wherein the fiber coating is applied to both sides of the layer.

3. The method of claim 1, wherein the water-soluble fibers are produced by an electrospinning system.

4. The method of claim 1, wherein the fibers are composed of polyvinyl alcohol (PVA).

5. The method of claim 3, wherein the fibers are composed of polyvinyl alcohol (PVA).

6. The method of claim 2, wherein said layer of powder coating is discontinuous.

7. The method of claim 1, wherein more than one layer of said fine water-soluble fibers are applied to one or both sides of said layer of medicament powder.

8. The method of claim 1, wherein said medicament comprises aspirin.

9. The method of claim 1, wherein said fine water-soluble fibers are animal ingestible.

10. The method of claim 9, wherein said animal is a human.

11. A handleable wafer of medicament, comprising
(a) a medicament in the form of a layer of powder; and
(b) a layer of fine water soluble fibers disposed on at least one side of the powder layer, said fibers having diameters of between 50 nanometers and 20 micrometers.

12. The handleable wafer of medicament of claim 11, wherein the fiber layer is disposed both sides of the layer of powder.

13. The handleable wafer of medicament of claim 11, wherein the water-soluble fibers are produced by an electrospinning system.

14. The handleable wafer of medicament of claim 11, wherein the fibers are composed of polyvinyl alcohol (PVA).

15. The handleable wafer of medicament of claim 13, wherein the fibers are composed of polyvinyl alcohol (PVA).

16. The handleable wafer of medicament of claim 11, wherein said layer of powder coating is discontinuous.

17. The handleable wafer of medicament of claim 11, wherein more than one layer of said fine water-soluble fibers are applied to one or both sides of said layer of medicament powder.

18. The handleable wafer of medicament of claim 11, wherein said medicament comprises aspirin

19. The handleable wafer of medicament of claim 11, wherein said water-soluble fibers are animal ingestible.

20. The handleable wafer of medicament of claim 19, wherein said animal is a human.
